(19) United States
(12) Patent Application Publication
(10) Pub. No.: US 2023/0214996 A1
(43) Pub. Date: Jul. 6, 2023

(54) EYES MEASUREMENT SYSTEM, METHOD AND COMPUTER-READABLE MEDIUM THEREOF

(71) Applicant: National Yang Ming Chiao Tung University, Taipei (TW)

(72) Inventors: Oscar Kuang-Sheng Lee, Taipei (TW); Hung-Chang Chen, Taipei (TW); Si-Han Wang, Taipei (TW)

(21) Appl. No.: 17/725,578

(22) Filed: Apr. 21, 2022

Related U.S. Application Data
(60) Provisional application No. 63/294,924, filed on Dec. 30, 2021.

(51) Int. Cl. G06T 7/00; G06T 7/70; G16H 30/40; G16H 50/30

(52) U.S. Cl. CPC G06T 7/0012; G06T 7/70; G06T 7/97; G16H 30/40; G16H 50/30; G06T 2207/30041; G06T 2207/20081

(57) ABSTRACT

An eyes measurement system, a method and a computer-readable medium are provided, including a client device with a measurement application and a cloud processing device, where the cloud processing device receives the subject's eye images uploaded by the measurement application. After pre-processing the eye images, the cloud processing device uses a prediction model to obtain the predicted eye measure of the subject such as an MRD1, an MRD2 and an LF, and presents the predicted eye measure of the MRD1, the MRD2 and the LF to the clinicians as a basis for diagnosis. Therefore, the eye images are taken without restricting to the places, and the prediction model is used to accurately obtain the subject's eye measure, thereby providing the clinicians with a clear basis for diagnosis.

### BACKGROUND

### 1. Technical Field

The present disclosure relates to a technology of measurement, and more particularly, to an eyes measurement system, a method, and a computer-readable medium thereof.

### 2. Description of Related Art

In the prior arts, before performing eye surgeries or during diagnosing, the clinicians conducted eye measurements manually. For example, the clinicians set up a camera in front of the patient, keep the camera horizontal at the patient's eyes level, and maintain a fixed distance between the camera and the patient in order to take photographs of the patient's eyes. After that, the clinicians manually obtain various measures of the eyes according to the patient's eye photographs.

As can be seen in the prior art above, the measurement of the subject's eyes is conducted in a conventional and inconvenient way and is confined to the environment and method of taking photographs, which is not only difficult for obtaining such photographs, but also leads to inaccuracy of the manually conducted eye measure due to man-made errors during taking photographs. This would lead to serious misjudgments by the clinicians while making their diagnosis.

Therefore, how to conduct an eye measurement that obtains a subject's eye measure accurately and quickly without restricting by the environment and to provide a clear diagnosis basis for the clinicians has become an urgent issue in the art.

### SUMMARY

In order to solve and benefit the above-mentioned conventional technical problems, the present disclosure provides an eye measurement system, which includes: a client device with a measurement application, configured for capturing a first eye image, a second eye image and a third eye image of a subject, or for selecting the first eye image, the second eye image and the third eye image of the subject by the measurement application from the client device; and a cloud processing device, communicatively connected to the client device, configured for receiving the first eye image, the second eye image and the third eye image of the subject from the measurement application, and the cloud processing device includes: a pre-processing module, configured for cropping the first eye image, the second eye image and the third eye image of the subject into a first eye orbit image, a second eye orbit image and a third eye orbit image, respectively, and superimposing the second eye orbit image to the third eye orbit image to generate a superimposed eye orbit image; and an eye measure prediction module with a prediction model to calculate a predicted eye measure of the subject according to the first eye orbit image and the superimposed eye orbit image of the subject, wherein the cloud processing device sends the predicted eye measure of the subject back to the measurement application for supplying the predicted eye measure.

The present disclosure further provides an eye measurement method, comprising: capturing a first eye image, a second eye image, and a third eye image of a subject by a client device with a measurement application, or selecting the first eye image, the second eye image, and the third eye image of the subject from the client device by the measurement application; receiving the first eye image, the second eye image and the third eye image of the subject from the measurement application by a cloud processing device; cropping the first eye image, the second eye image and the third eye image of the subject into a first eye orbit image, a second eye orbit image, and a third eye orbit image by the cloud processing device, respectively, wherein the second eye orbit image is superimposed to the third eye orbit image to generate a superimposed eye orbit image; using a prediction model by the cloud processing device to calculate a predicted eye measure of the subject based on the first eye orbit image and the superimposed eye orbit image; and sending the predicted eye measure of the subject back to the measurement application by the cloud processing device for supplying the predicted eye measure.

In the aforementioned embodiment, the first eye image is a photograph of the subject's left and right eyes viewing forward, the second eye image is a photograph of the subject's left and right eyes gazing up, and the third eye image is a photograph of the subject's left and right eyes gazing down.

In the aforementioned embodiment, the predicted eye measure includes an MRD1 (Margin Reflex Distance 1, a vertical distance between an upper eyelid margin and a corneal light reflex), an MRD2 (Margin Reflex Distance 2, a vertical distance between a lower eyelid margin and the corneal light reflex) and an LF (Levator Function), wherein the MRD1 and the MRD2 are calculated from the first eye orbit image, and the LF is calculated from the superimposed eye orbit image by the prediction model of the eye measure prediction module.

In the aforementioned embodiment, the pre-processing module uses a coordinate regression model to determine a position of a corneal light reflex of the first eye image, the second eye image and the third eye image of the subject, and crops the first eye image, the second eye image, and the third eye image into the first eye orbit image, the second eye orbit image and the third eye orbit image based on the position of the corneal light reflex.

In the aforementioned embodiment, the measurement application includes a data management module for viewing the subject's data, eye operation information, or monitoring subject's eye condition.

In the aforementioned embodiment, the measurement application includes a notification management module for receiving health-related information and notifications, managing the subject's groups, and displaying sent message history.

In the aforementioned embodiment, the prediction model is established based on an EfficientNet in combination with a SENet, the eye measure prediction module inputs a plurality of training images into the prediction model to perform deep learning, and the prediction model calculates the predicted eye measure of the subject based on the first eye orbit image and the superimposed eye orbit image after finishing the deep learning, wherein the SENet increases feature weights of significant features and reduces feature weights of invalid or insignificant features in the plurality of training images, such that the EfficientNet performs the deep learning based on adjusted feature weights and the plurality of training images.

The present disclosure further provides a computer-readable medium for use in a computer or computing device having a processor and/or a memory, wherein the computer or computing device executes a target program and a computer-readable medium via the processor and/or the memory for performing the above-described eye measurement method while executing the computer-readable medium.

As can be seen from the above, the eye measurement system, the eye measurement method, and the computer-readable medium of the present disclosure receives the first eye image, the second eye image, and the third eye image of the subject from the uploads by clinicians or subjects using a measurement application in a client device via a cloud processing device. After the first eye image, the second eye image and the third eye image are pre-processed, the first eye orbit image and the superimposed eye orbit image of the patient are obtained, such that the prediction model can obtain the MRD1, the MRD2, and the LF of the patient based on the first eye orbit image and the superimposed eye orbit image, and provide the MRD1, the MRD2 and the LF of the patient to the clinicians as the basis for diagnosis. Therefore, compared with the prior art, the present disclosure does not confine to the places where the eye images are taken, and the prediction model is used to accurately obtain the subject's eye measure, thereby providing the clinicians with a clear basis for diagnosis.

### BRIEF DESCRIPTION OF THE DRAWINGS

FIG. 1 is a schematic architecture diagram illustrating the eye measurement system of the present disclosure.

FIG. 2 is a detailed functional architecture diagram of the measurement application.

FIG. 2-1 to FIG. 2-34 are schematic diagrams of the user interface of the measurement application.

FIG. 3-1 is a diagram illustrating the first eye image of a subject.

FIG. 3-2 is a diagram illustrating the first eye orbit image of a subject.

FIG. 3-3 is a diagram illustrating the second eye image of a subject.

FIG. 3-4 is a diagram illustrating the second eye orbit image of a subject.

FIG. 3-5 is a diagram illustrating the third eye image of a subject.

FIG. 3-6 is a diagram illustrating the third eye orbit image of a subject.

FIG. 3-7 is a diagram illustrating the superimposed eye orbit image of a subject.

FIG. 4 is a flowchart illustrating the training of the prediction model of the eye measure prediction module.

FIG. 4-1 is an architecture diagram illustrating the prediction model.

FIG. 5-1 is a diagram illustrating MRD1.

FIG. 5-2 is a diagram illustrating MRD2.

FIG. 5-3 is a diagram illustrating the LF.

FIG. 6 is a flowchart illustrating the eye measurement method of the present disclosure.

### DETAILED DESCRIPTION

The following describes the implementation of the present disclosure through embodiments, and those skilled in the art can easily understand other advantages and effects of the present disclosure from the contents disclosed in this specification. It should be noted that the structures, proportions, sizes, etc. shown in the accompanying drawings in this specification are used to cooperate with the contents disclosed in the specification, for the understanding and reading of those who are familiar with this field, and are not used to limit this specification. Any modification of the structure, change of the proportional relationship or adjustment of the size should not affect the effect and the purpose of the present disclosure, and it falls within the scope of the technical content disclosed in the present disclosure. At the same time, terms such as “a,” “first,” “second,” “upper” and “lower” quoted in this specification are for the convenience of description and are not used to limit the scope of the present disclosure. The scope of implementation and the change or adjustment of its relative relationship shall be regarded as the scope of implementation of the present disclosure without substantially changing the technical content.

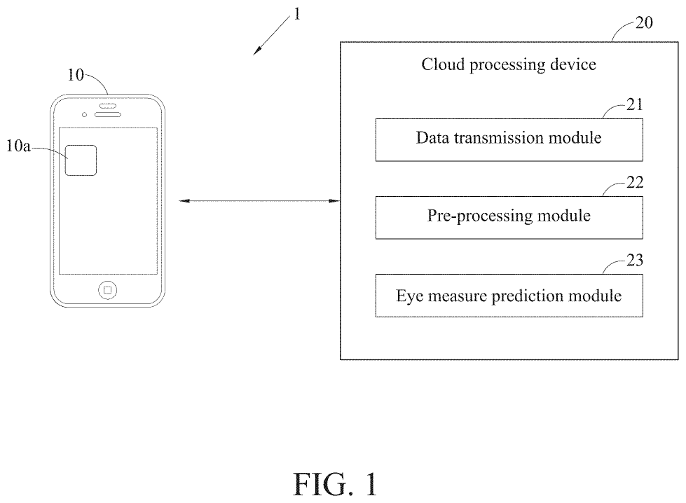

FIG. 1 is a schematic architecture diagram illustrating the eye measurement system 1 of the present disclosure. As shown in FIG. 1, the eye measurement system 1 includes a client device 10 having a measurement application 10a and a cloud processing device 20, wherein the cloud processing device 20 includes a data transmission module 21, a pre-processing module 22, and an eye measure prediction module 23. In addition, the client device 10 communicates with the measurement device 20 via wired or wireless communication technology, such as cellular network, mobile network (4G, 5G, 6G, etc.), Wi-Fi or Bluetooth.

In one embodiment, the client device 10 includes, but is not limited to, a smartphone, a personal computer, a notebook computer or other electronic devices with computing capabilities, and the electronic device includes a processor, a display screen, a camera lens, etc. The measurement application 10a is executed via the processor of the client device 10, and the measurement application 10a can be an application (APP) installed on a smartphone. Furthermore, the cloud processing device 20 can be built in the same (or different) server (such as a general-purpose server, file-type server, a storage-unit-type server, etc.) and an electronic device with a suitable computing mechanism, such as a computer, wherein each module in the client device 10 and the cloud processing device 20 can be software, a hardware or a firmware; if it is a hardware, it can be a processing unit, processor, computer or server with data processing and computing capabilities; in the case of software or firmware, it may include instructions executable by a processing unit, processor, computer or server, and may be installed on the same hardware device or distributed across multiple hardware devices.

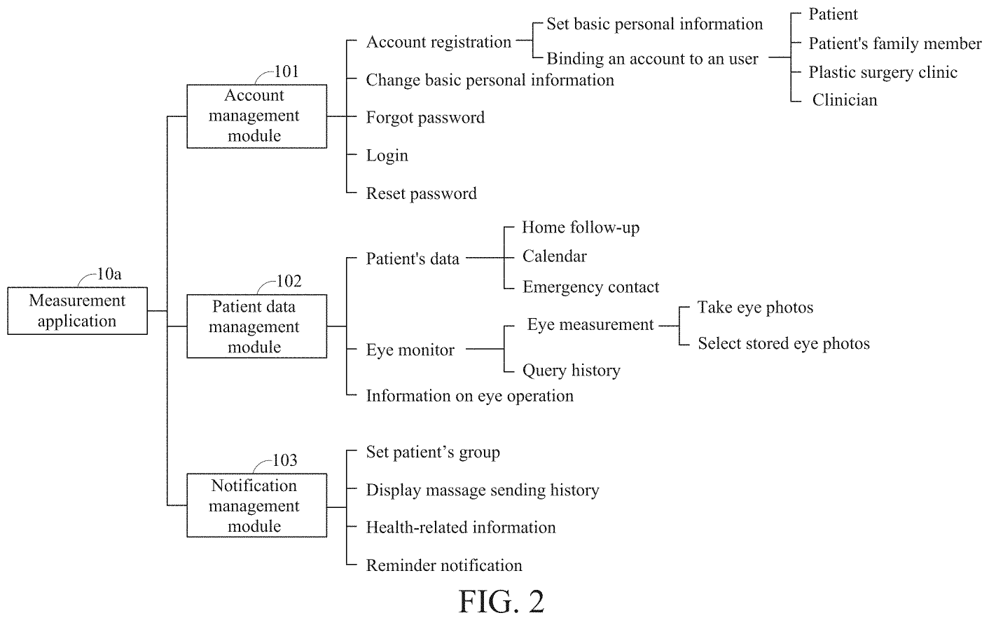

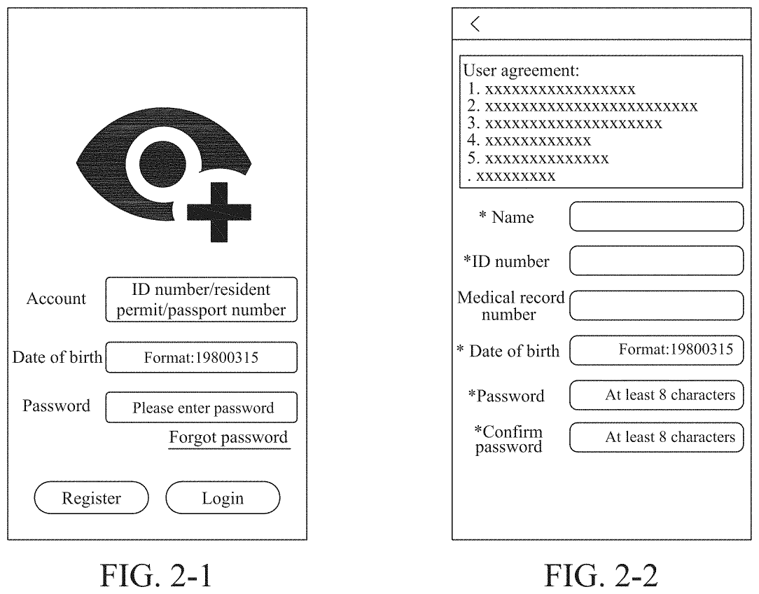

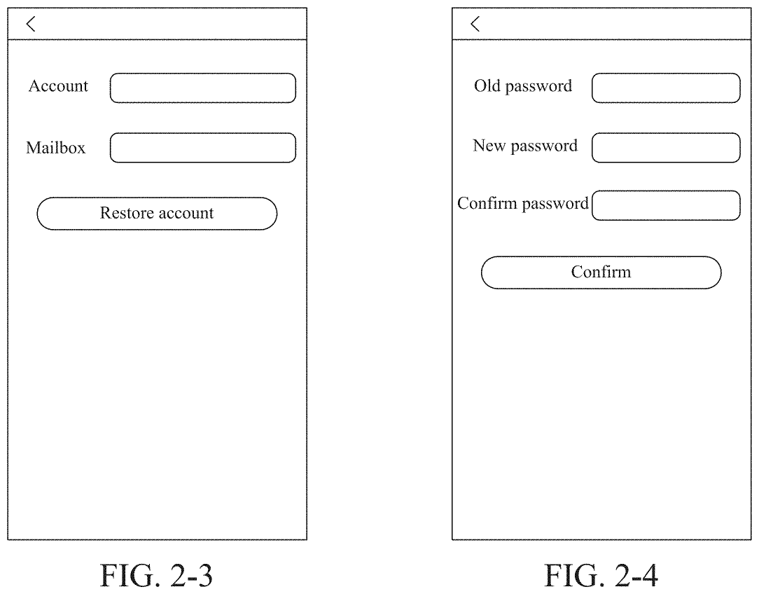

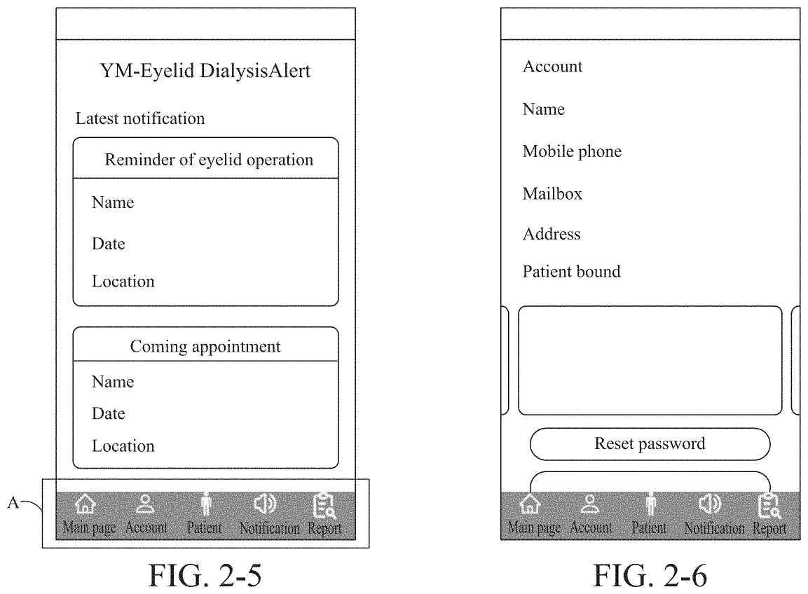

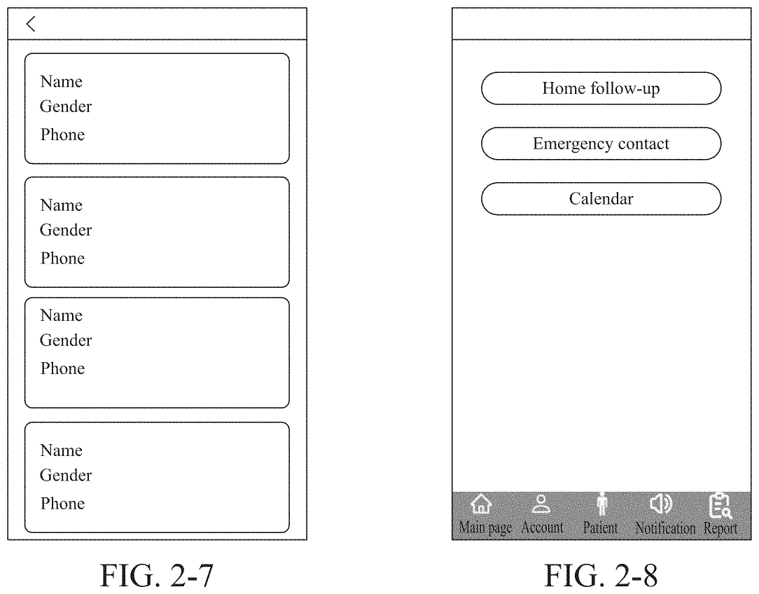

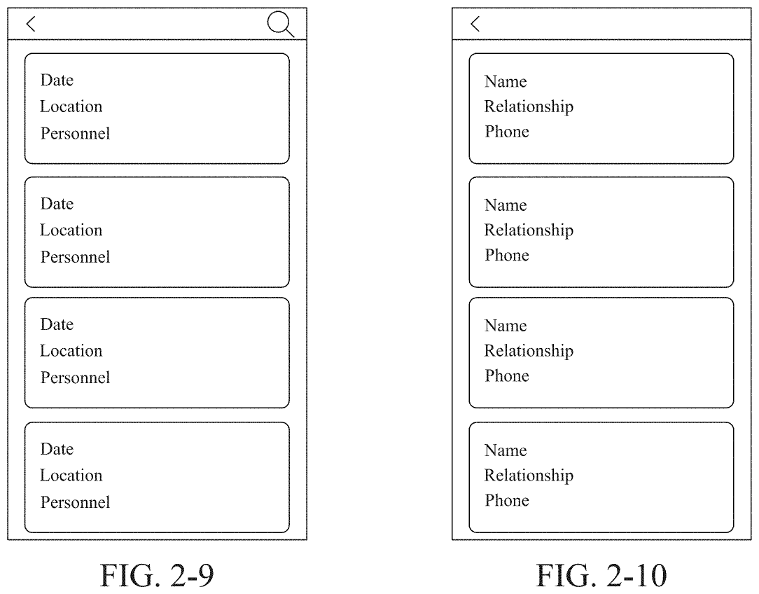

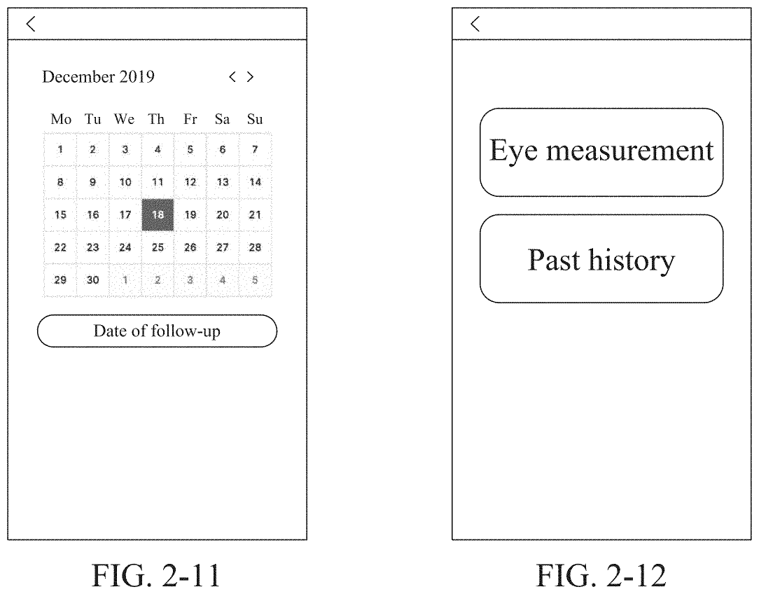

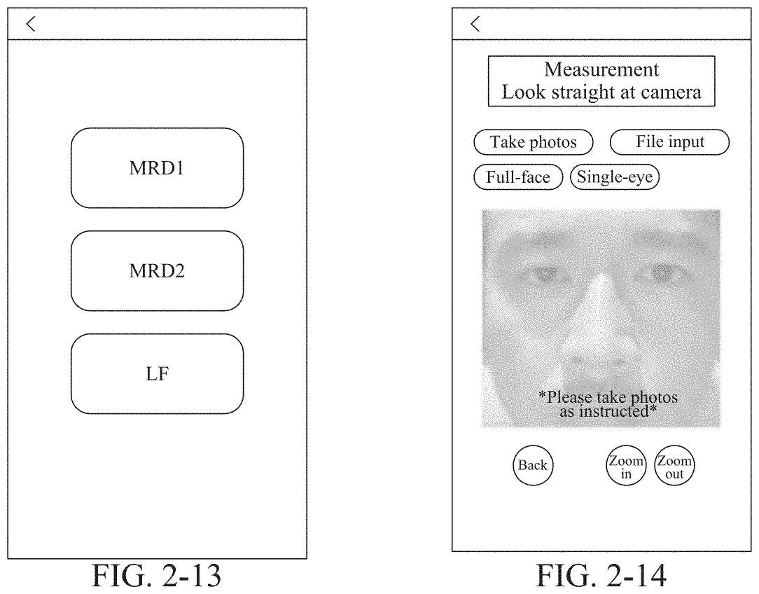

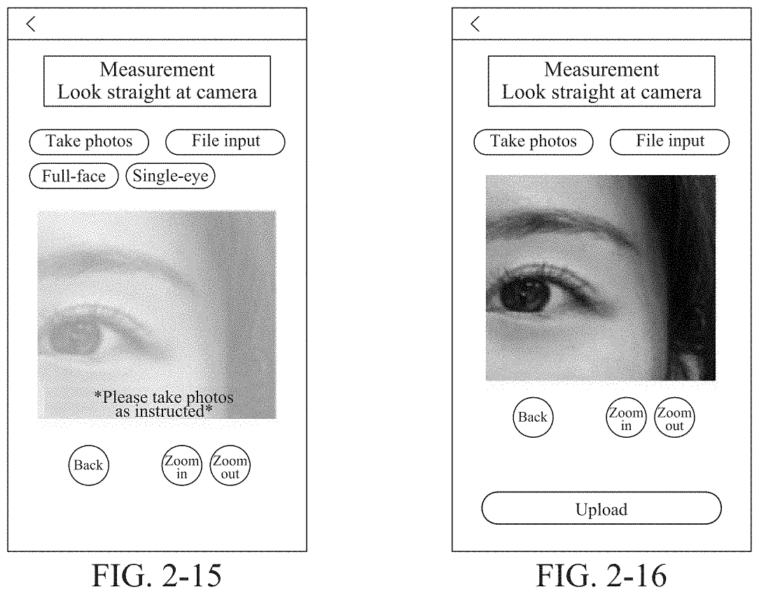

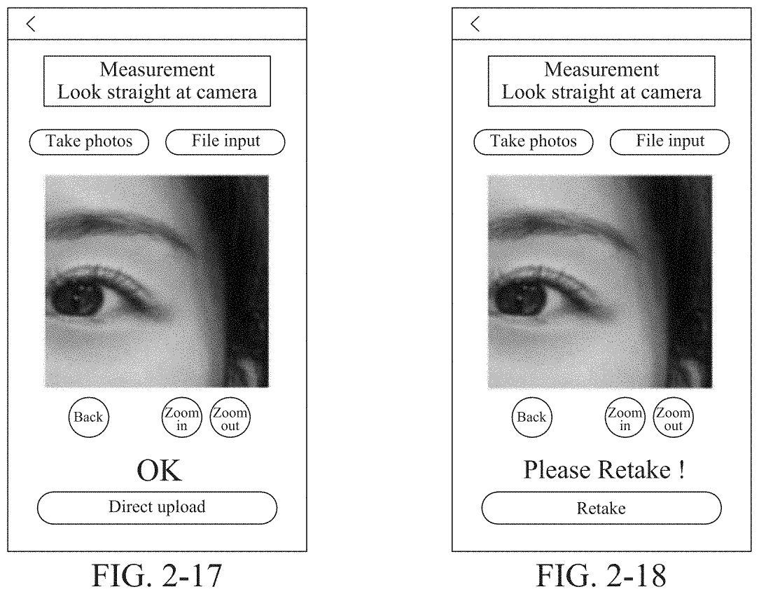

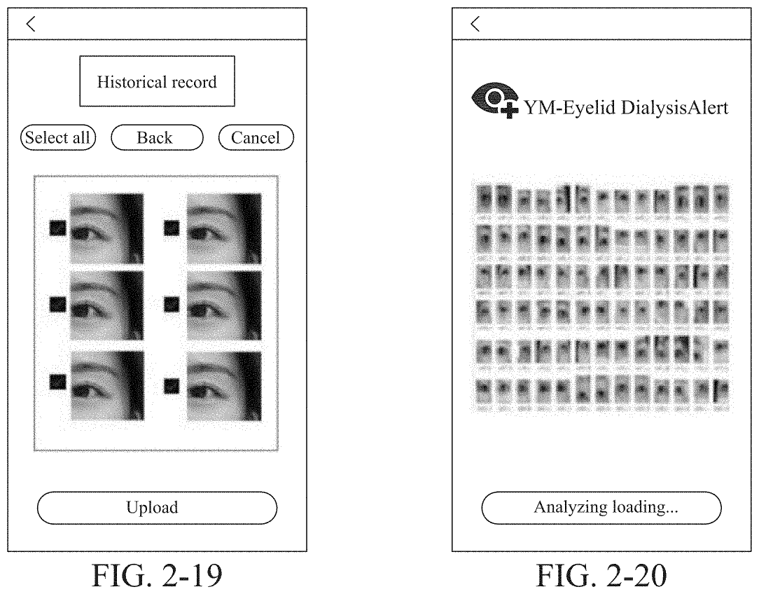

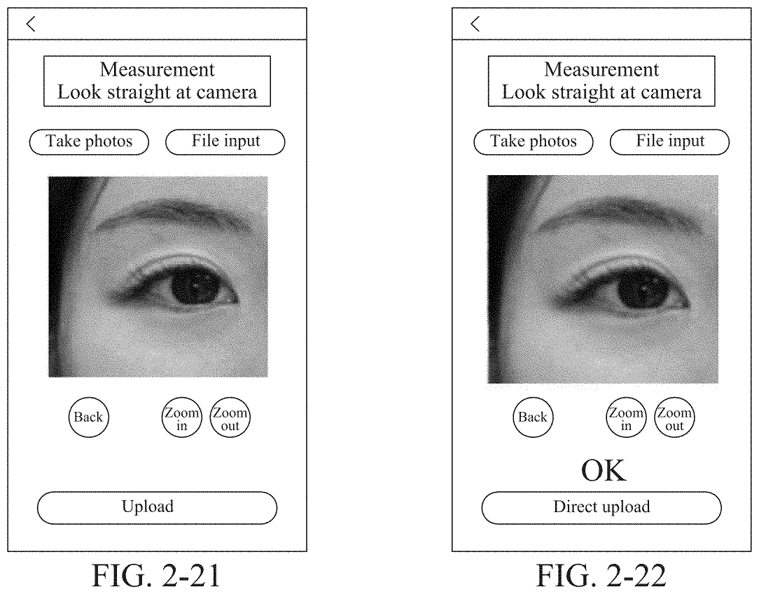

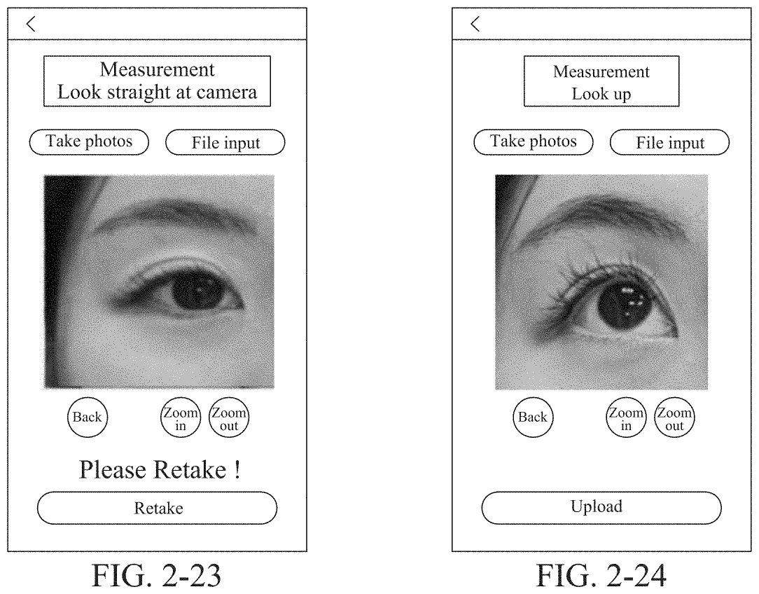

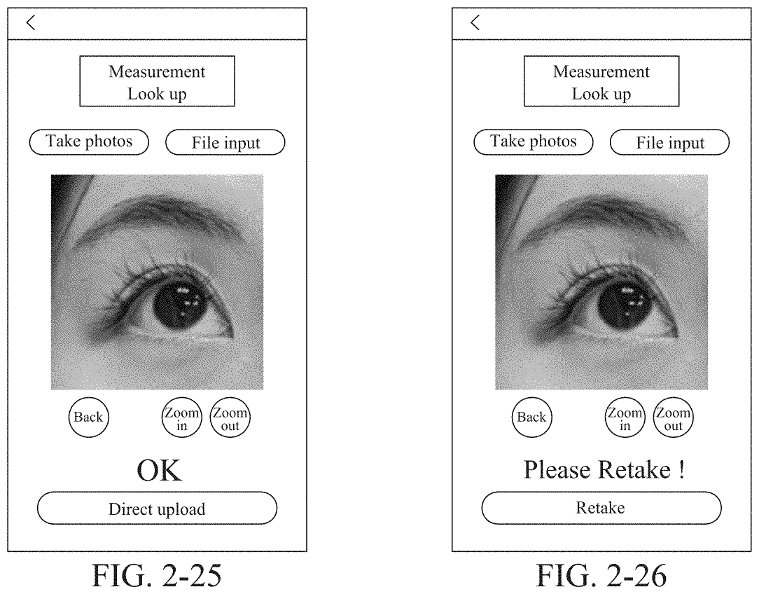

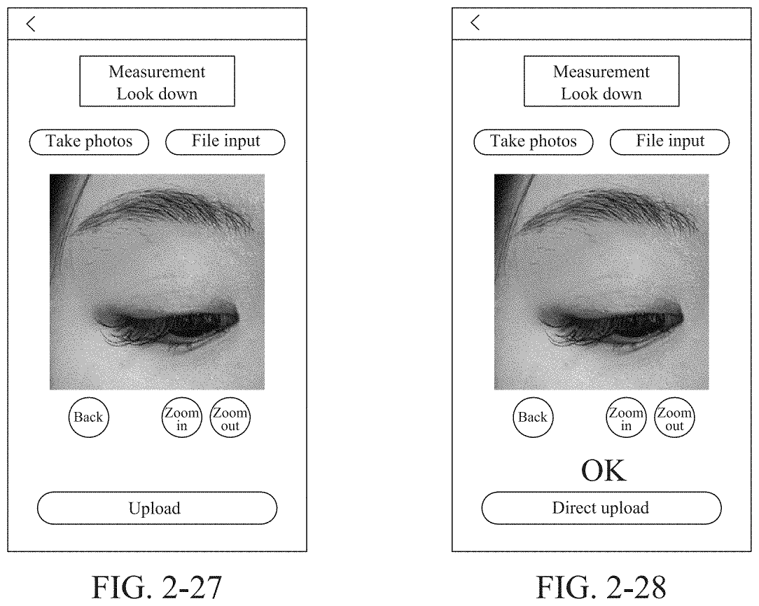

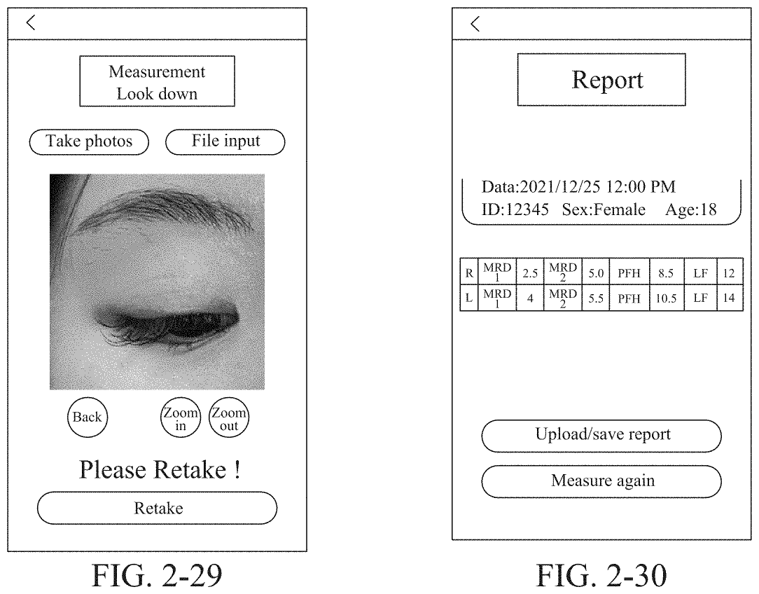

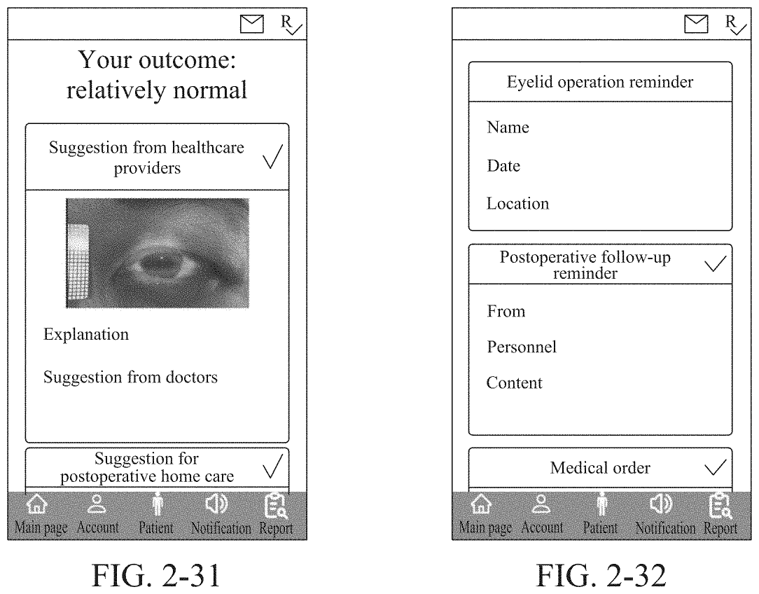

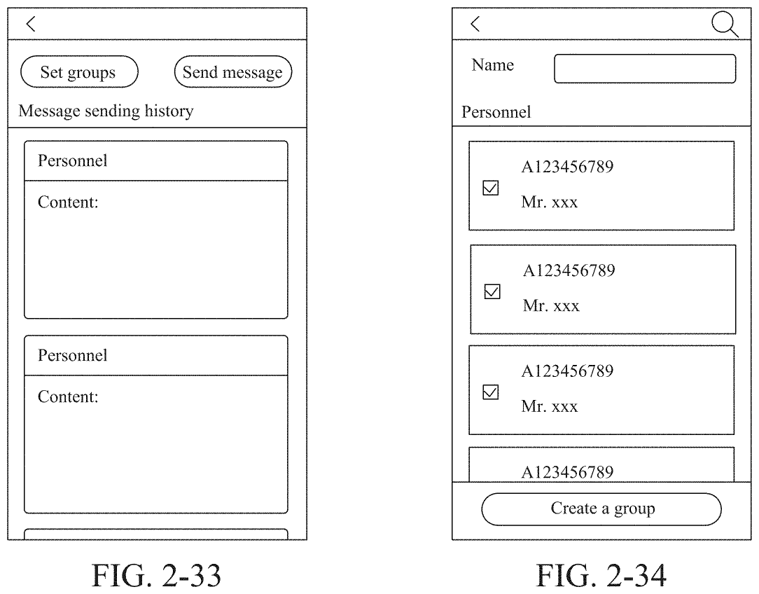

FIG. 2 is a detailed functional architecture diagram of the measurement application 10a. As shown in FIG. 2, a measurement application 10a is installed in the client device 10, and the measurement application 10a includes an account management module 101, a patient data management module 102, and a notification management module 103, wherein the measurement application 10a is displayed on the display screen of the client device 10 via a user interface (UI), so that the account management module 101, the patient data management module 102 and the notification management module 103 provide a user eye measurement related service.

As shown in FIG. 2-1 to FIG. 2-4, when the user starts the measurement application 10a, the account management module 101 displays a login interface (as shown in FIG. 2-1), so that the user can input user account (such as ID card number, residence permit number or passport number, etc.), date of birth and password, etc., to login to the measurement application 10a.

In one embodiment, when the user is a new user, the account management module 101 allows the user to click the “register” button in the login interface (as shown in FIG. 2-1) and leads the user to read the terms of use in the registration interface (as shown in FIG. 2-2), and sets the basic personal information of the user (such as name, ID number, medical record number, date of birth, login password, confirmation password, mailbox [such as e-mail] or mobile phone number, etc., which are not limited herein), and then enables the account management module 101 for the user to register a new account and binds the new account to the specific user. In another embodiment, when the account management module 101 is for the user to register a new account, the account management module 101 is for the user to set his/hers identity, such as: the patient himself/herself, the patient's family member, a clinician, or a plastic surgery clinic etc., so that the account management module 101 confirms that the user is the patient himself/herself, a patient's family member, a clinician, or a plastic surgery clinic according to the user account, date of birth and password entered by the user.

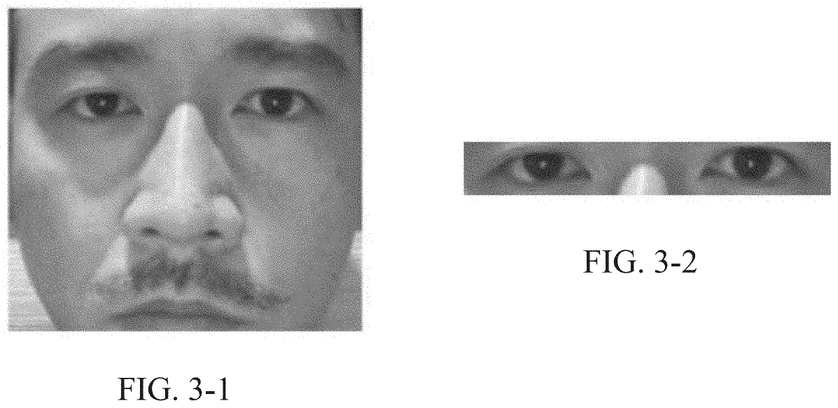

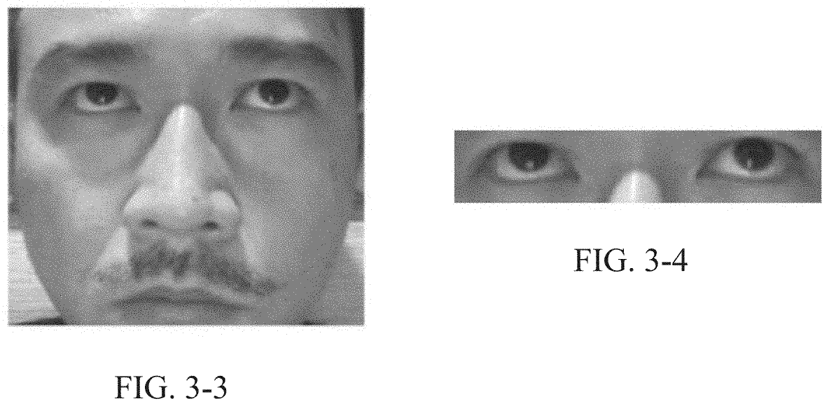

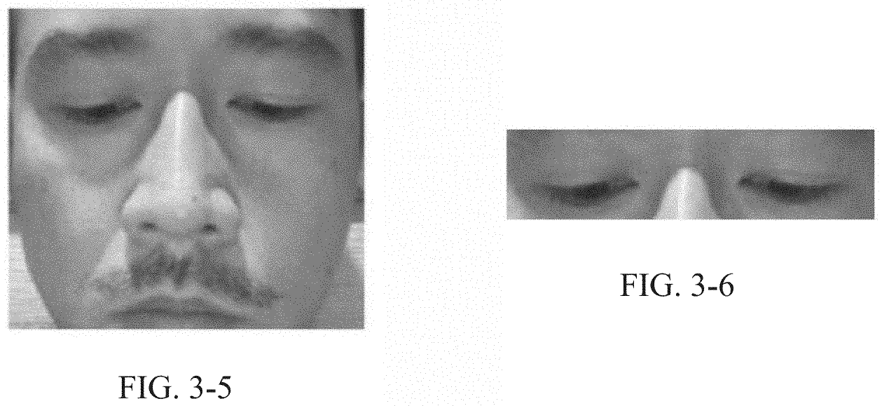

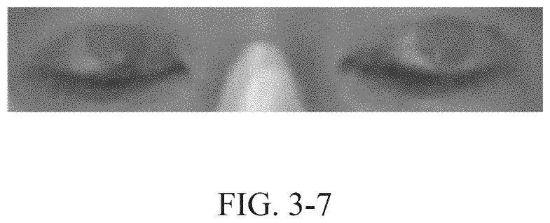

In one embodiment, when the user forgets the password, the account management module 101 allows the user to click the “forgot password” button in the login interface (as shown in FIG. 2-1), so that after the user entering the account ID and mail address (e.g., e-mail) in the account restoration interface (as shown in FIG. 2-3), the account management module 101 sends the account reset link by e-mail to enable the user to reset the password. In another embodiment, the account management module 101 can also be used for the user to input a mobile phone number via the account restoration interface, and to perform the user's identity verification via the OTP (One Time Password) verification method. When the terminal device 10 receives the OTP password, the user enters the OTP password in the account restoration interface, so that the account management module 101 confirms the user's identity, and then redirects to the password reset interface (as shown in FIG. 2-4) for the user to reset the password, and the present disclosure does not limit the way in which the user password is reset.

In one embodiment, when the user needs to reset the password, the account management module 101 provides the password reset interface (as shown in FIG. 2-4) for the user to reset the password via entering the old password, new password and confirming password to finish the password reset process.

In one embodiment, when the user modifies his/her basic personal information, the account management module 101 provides a data change interface (as shown in registration interface of FIG. 2-2) for the user to edit their basic personal information in the data change interface, and basic personal information editing is completed after clicking Save Account Basic Information.

As shown in FIGS. 2-5 to 2-31, after the user logins to the measurement application 10a, the patient data management module 102 displays a main page with a menu A (as shown in FIG. 2-5), and the main page contains eye operation information, wherein the eye operation information includes but is not limited to eye operation date reminder and/or notification for the coming appointment, and the eye operation date reminder and/or the notification for the coming appointment includes the user's name, date and/or location, and other information, so as to provide the user with a reminder of the date of operation and the coming appointment. In addition, the patient data management module 102 allows the user to click the “Home” button in the menu A to return to the main page interface.

In one embodiment, when the user is viewing his/her basic personal information, the patient information management module 102 allows the user to click the “account” button in menu A of the main page, and then the patient information management module 102 displays a basic personal information interface (as shown in FIG. 2-6) to display the user's basic personal information (such as account ID, name, mobile phone number, etc.) to the user. In another embodiment, if the identity of the user is a clinician or a plastic surgery clinic instead of a patient, when the user views his/her basic personal information, after clicking the “Account” button in menu A of the main page, the patient data management module 102 displays a patients' basic personal information interface (as shown in FIG. 2-7), such that the basic personal information (such as name, gender, mobile phone number, etc.) of multiple patients are presented to the user.

In one embodiment, when the user inquires about his/her patients' information, the patient information management module 102 allows the user to click the “patient” button in menu A of the main page interface, and then the patient information management module 102 displays a patients' information interface (as shown in FIG. 2-8) for presenting data such as home follow-up, emergency contact and/or calendar to the user.

In one embodiment, after clicking the “Home follow-up” button in the patient information interface, the patient information management module 102 displays the preoperative and/or postoperative test results list of the user (i.e., patient) (as shown in FIG. 2-9) which includes but is not limited to the location, date, and personnel of the test. After clicking the “emergency contact” button in the patient information interface, the patient information management module 102 displays the emergency contact list of the user (i.e., patient) (as shown in FIG. 2-10) which includes but is not limited to name, relationship and phone number. After clicking the “emergency contact” button in the patient information interface, the patient data management module 102 displays the calendar of the user (i.e., patient) (as shown in FIG. 2-11), and connects to the commonly used calendar scheduling software (e.g., google calendar) in the client device 10 (e.g., a smart phone). According to the list of measurements, the patient information management module 102 sets a reminder for measurement across a specific time span, and uses color to highlight the specific dates in the calendar, where different appointments are highlighted with different colors (e.g., blue for upcoming measurements, etc.). Therefore, the calendar allows the patients and healthcare providers to know the schedule of the measurement and medical appointments quickly.

In one embodiment, when the user needs to perform eye monitoring, the patient data management module 102 allows the user to click the “Report” button in menu A of the main page, and then the patient data management module 102 displays a main monitoring interface (as shown in FIG. 2-12) for the user to choose to perform eye measurement after clicking the button of “eye measurement” or “past history” in the main monitoring interface, wherein the patient data management module 102 displays a past data interface (as shown in FIG. 2-19) which includes all of the past predicted eye measures (including MRD1, MRD2 and LF) and the patient's medical records and interview profiles.

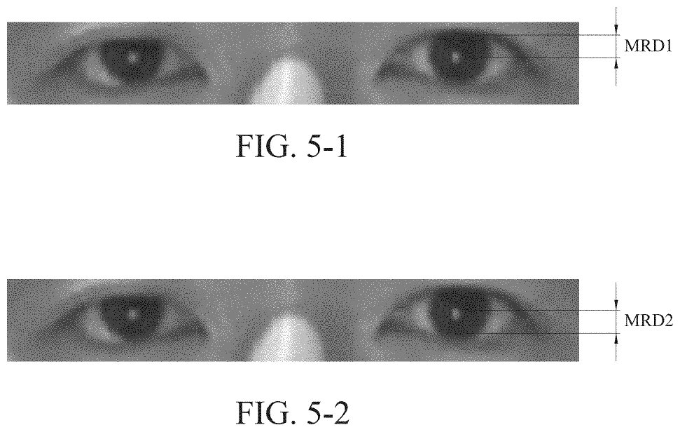

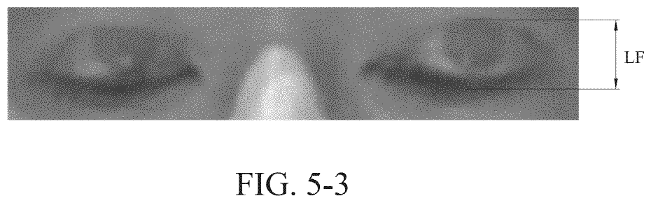

Furthermore, after the user clicks “eye measurement” in the main monitoring interface, the patient data management module 102 displays a measurement sub-interface (as shown in FIG. 2-13) for the user to choose from measuring MRD1, MRD2 or LF, and among which, MRD1 is the distance between the center of the pupil in the eye image to the margin of the upper eyelid (as shown in FIG. 5-1), MRD2 is the distance between the center of the pupil in the eye image and the margin of the lower eyelid (as shown in FIG. 5-2), and LF is the distance of the upper eyelid movement between the image of the eye gazing up and the image of the eye gazing down (as shown in the FIG. 5-3).

In one embodiment, when measuring MRD1, the patient data management module 102 allows the user to click the button of “MRD1” in the measurement sub-interface to display an MRD1 measurement interface (as shown in FIG. 2-14), for the user to choose to use the client device to capture the first eye image of the left eye and/or the right eye, or to select the left eye in the client device 10 (such as a smartphone). The first eye image of the left eye and/or the right eye is sent to the patient data management module 102, and the first eye image in the MRD1 measurement interface can further be zoomed in and out, as well as to retake the image. It should be noted that the first eye image is an image of the left eye and/or the right eye of the user (i.e., patient) viewing forward.

Furthermore, the MRD1 measurement interface is for the user to click the button of “Full-face” and then choose to take photos of the full-face area of the user (as shown in FIG. 2-14), or for the user to click the “Single-eye” button and then select to take photos of the user's monocular area (as shown in FIG. 2-15). It should be noted that, for full-face photos, at least one eye image (including the left and right eyes) is required, while for monocular photography, at least one eye image for each of the left eye and/or the right eye is required.

After the patient data management module 102 obtains the user's first eye image, the patient data management module 102 allows the user to click the “file input” button to display a data upload interface (as shown in FIG. 2-16), allowing the user to confirm the first eye image. Then, the patient information management module 102 allows the user to click the “upload” button. After the patient information management module 102 confirms that the first eye image meets the standard, the data upload interface displays “OK” (as shown in FIG. 2-17) for the user to click the “Direct upload” button to upload the first eye image to the cloud processing device 20. For instance, the first eye image meets the standard when it does not have unnatural flash points, blurriness, skewed angles, or problems that the subject in the photo is not an eye. After that, the cloud processing device 20 calculates the user's first eye image to measure MRD1, and the patient data management module 102 displays an analysis interface (as shown in FIG. 2-20) to inform the user that MRD1 measurement has been performed. On the contrary, if the patient data management module 102 confirms that the first eye image does not meet the standard, the data upload interface will display “Please Retake” (as shown in FIG. 2-18) to remind the user to retake or select the first eye image.

In another embodiment, the measurement of MRD2 is substantially the same as the measurement of MRD1 in the above-mentioned embodiment. When measuring MRD2, the patient data management module 102 allows the user to click the button “MRD2” in the measurement sub-interface to display an MRD2 measurement interface (not shown) managed by the patient data module 102, and is for the user to choose to use the client device to capture the first eye image of the left eye and/or the right eye, or to select the left eye and/or the right eye from the client device 10 (such as a smartphone). The first eye image is sent to the patient data management module 102.

After the patient data management module 102 received the user's first eye image, the patient data management module 102 allows the user to click the “file input” button to display a data upload interface (as shown in FIG. 2-21) for the user to confirm the first eye image. Then, the patient information management module 102 allows the user to click the “upload” button. After the patient information management module 102 confirms that the first eye image meets the standard, the data upload interface displays “OK” (as shown in FIG. 2-22), allowing the user to click the “Direct upload” button to upload the first eye image to the cloud processing device 20. For instance, the first eye image meets the standard when it does not have unnatural flash points, blurriness, skewed angles, or problems that the subject in the photo is not an eye. After that, the cloud processing device 20 performs operations on the user's first eye image to measure MRD2, and the patient data management module 102 displays an analysis interface (as shown in FIG. 2-20) to inform the user that MRD2 measurement has been performed. On the contrary, if the patient data management module 102 confirms that the first eye image does not meet the standard, the data upload interface displays “Please Retake” (as shown in FIG. 2-23), reminding the user to retake or select the first eye image. In another embodiment, the measurement of LF is substantially the same as the measurement of MRD1 in the above-mentioned embodiment. When measuring LF, the patient data management module 102 allows the user to click the button “LF” in the measurement sub-interface to display a LF measurement interface (not shown) for the user to select the client device to capture the second eye image and the third eye image of the left eye and/or the right eye, or select the left eye and/or the right eye from the client device 10 (such as a smartphone). The second eye image and the third eye image of the eye are sent to the patient data management module 102, and the LF measurement interface also has a function for enlarging and minimizing the second eye image and the third eye image, as well as the function to return to retake the second eye image and the third eye image. It should be noted that the second eye image is the image of the left eye and/or the right eye of the user (i.e., patient) while gazing up, and the third eye image is the image of the left eye and/or the right eye gazing down. In addition, the LF measurement interface also allows the user to click “Full-face” button to choose to capture the user's whole face (not shown), or for the user to click the “Single-eye” button to choose to capture the user's monocular area (not shown). It should be noted that for full-face picture, at least two eye images (looking up and looking down) are required, and for monocular picture, at least two eye images are required for each of the left eye and/or the right eye (gazing up and down).

After the patient data management module 102 obtains the second eye image and the third eye image of the user, the patient data management module 102 allows the user to click “file input” button to display a data upload interface (as shown in FIG. 2-24 and FIG. 2-27) for the user to confirm the second eye image and the third eye image. Then, the patient data management module 102 allows the user to click “upload” button. After the patient data management module 102 confirms that the image of the second eye and the image of the third eye meet the standard, the data upload interface shows “OK” (as shown in FIG. 2-25 and FIG. 2-28) and allows the user to click “Direct upload” button to upload the second eye image and the third eye image to the cloud processing device 20. For instance, the second eye image and the third eye image meet the standard when they do not have unnatural flash points, blurriness, skewed angles, or problems that the subject in the photo is not an eye. After that, the cloud processing device 20 calculates the second eye image and the third eye image of the user to measure LF, and the patient data management module 102 displays an analysis interface (as shown in FIG. 2-20) to inform the user that the LF measurement has been performed. On the contrary, if the patient data management module 102 confirms that the second eye image and the third eye image do not meet the standard, the data upload interface displays “Please Retake” (as shown in FIG. 2-26 and FIG. 2-29) to remind the user to re-shoot or select the second and third eye images. In addition, in the historical record interface shown in FIG. 2-19, the patient data management module 102 can also allow the user to select and upload a plurality of the first eye images, the second eye images and the third eye images to the cloud processing device 20 via the historical record interface, so that the cloud processing device 20 can calculate based on the plurality of first eye images of the user to measure MRD1 and MRD2, and calculate based on the second eye images and the third eye images of the user to measure LF.

In one embodiment, after the cloud processing device 20 completes the measurement of the predicted values (i.e., MRD1, MRD2 and LF), the cloud processing device 20 sends an analysis report back to the measurement application 10a, and the patient data management module 102 presents the analysis report to the user via an analysis report interface (as shown in FIG. 2-30), wherein the analysis report includes but is not limited to the test date, the user's identification code (such as ID number, etc.), gender, name and predicted measure which includes but is not limited to MRD1, MRD2 and LF.

Furthermore, the patient data management module 102 is for the user to click the “upload and save report” button in the analysis report interface to upload the analysis report to the cloud processing device 20 for storage, and a healthcare provider uses another client device to obtain the analysis report from the cloud processing device 20, and the other client device provides medical advice (such as explanations and suggestions from medical personnel and/or postoperative care) provided by the healthcare provider based on the analysis report. Home care advices are transmitted via the cloud processing device 20 to the measurement application 10a in the client device 10 of the user. After that, the patient data management module 102 of the measurement application 10a displays the medical advice (as shown in FIG. 2-31) from the healthcare provider to the user.

As shown in FIG. 2-32 to FIG. 2-34, when the user views the notification, the patient data management module 102 is for the user to click “Notification” button in the menu A of the main page. The notification management module 103 displays a notification interface (as shown in FIG. 2-32) to provide a reminder notification and a health-related information to the user. In addition, when the user is a patient, the reminder notification also includes eye measurement reminder, eye operation reminder, postoperative follow-up reminder and/or a doctor's advice, etc. The eye operation reminder includes preoperative and postoperative reminders to the patients and their family members, as shown in Table 1.

TABLE 1

| | |
|---|---|
| Notification to patients and their | Post-operative notification to the |
| family members before operation | patients and their family members |

| | | | |
|---|---|---|---|
| 1. | Preoperative MRD1, MRD2 | 1. | Postoperative MRD1, MRD2 |
| | and LF | | and LF |
| 2. | Medication or treatment | 2. | Please take pictures of the eye |
| | information | | every day |
| 3. | Others | 3. | Vital signs: blood |
| | | | pressure/heart rate (if |
| | | | unstable, regular medication |
| | | | reminder) |
| | | 4. | Doctor's suggestion: |
| | | | Medication |
| | | | adjustment/massage/postoperative |
| | | | monitoring/outpatient |
| | | | follow-up/fistula care |
| | | 5. | Others |

Furthermore, when the user is a healthcare provider, the reminder notification also includes an eye operation reminder, etc., and the eye operation reminder includes preoperative and postoperative reminding to the healthcare provider, as shown in Table 2.

TABLE 2

| Notification to the healthcare | | Postoperative notification | |
|---|---|---|---|
| provider before operation | | to the healthcare provider | |
| 1. | Preoperative MRD1, MRD2 | 1. | Postoperative MRD1, MRD2 |
| | and LF | | and LF |
| 2. | Others | 2. | Postoperative home follow-up |
| | | | records |
| | | 3. | Others |

In another embodiment, when the user is a clinician or an orthopedic clinic, the notification management module 103 allows the user to click on “Letter” icon on the notification interface to display a message sending interface (as shown in FIG. 2-33) for the user (clinician) to view the messages sent to the patients, such as the reminder notice and the health-related information. Furthermore, the notification management module 103 is for the user (clinician) to click “group setting” in the message sending interface, so that the user (clinician) can categorize a plurality of patients into one specific patient group for sending health education information, etc. (as shown in FIG. 2-34). In addition, the notification management module 103 allows the user (clinician) to click “send message” on the message sending interface, so that the user (clinician) could send the notifications and the health-related information to the patient. As shown in FIG. 1, the data transmission module 21 in the cloud processing device 20 receives the first eye image, the second eye image and the third eye image uploaded by the measurement application 10a of the client device 10. The pre-processing module 22 in the cloud processing device 20 obtains the first eye orbit image, the second eye orbit image and the third eye orbit image based on the first eye image, the second eye image and the third eye image, wherein the first eye orbit image is primary-gaze orbital photographs, the second eye orbit image is a photograph of the subject's eyes gazing up, and the third eye orbit image is a photograph of the subject's eyes gazing down.

In one embodiment, refer to FIG. 3-1, FIG. 3-3 and FIG. 3-5, wherein FIG. 3-1 is the first eye image uploaded by the user (i.e., patient), FIG. 3-3 is the second eye image uploaded by the user (i.e., patient), and FIG. 3-5 is the third eye image uploaded by the user (i.e., patient). The pre-processing module 21 uses the label image to label the conical light reflex position in the first eye image, the second eye image and the third eye image, and the corneal light reflex position is presented in the form of coordinates (X, Y).

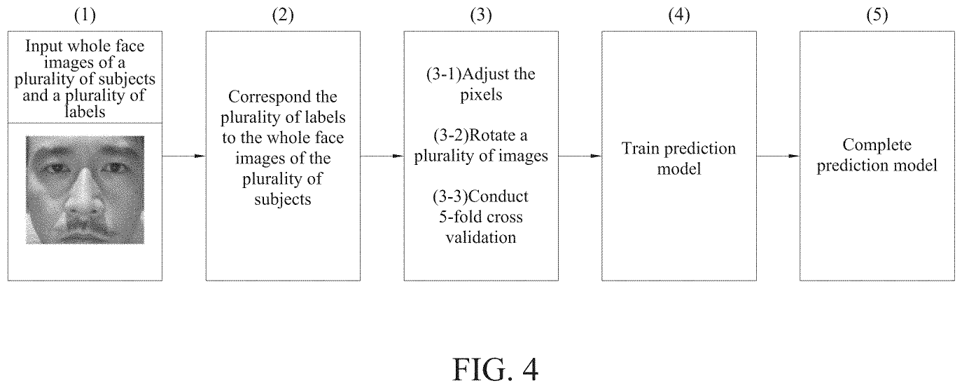

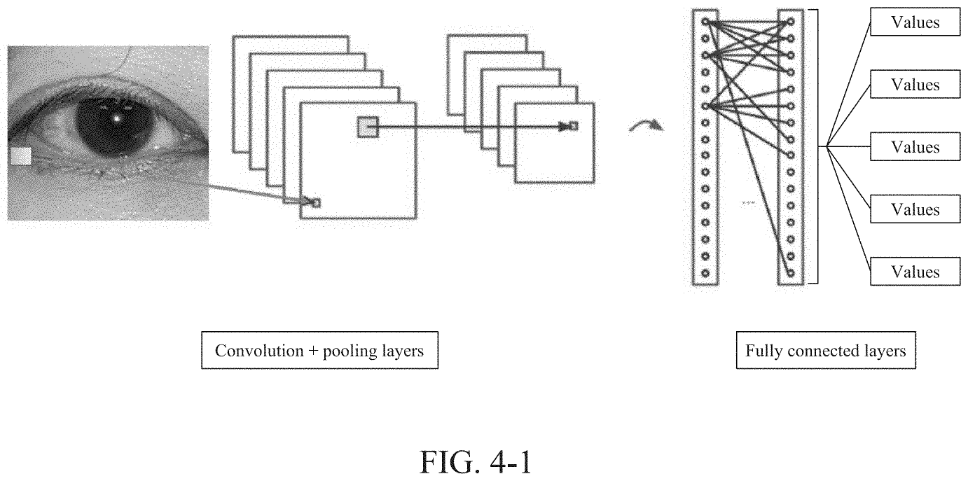

Furthermore, the pre-processing module 22 has a pupil coordinate regression model to determine the position of the corneal light reflex of the first eye image, the second eye image and the third eye image, respectively. After that, the pre-processing module 22 expands from the corneal light reflex position in the first eye image, the second eye image and the third eye image to automatically crop the images into the first eye orbit image (as shown in FIG. 3-2), the second eye orbit image (as shown in FIG. 3-4) and the third eye orbit image (as shown in FIG. 3-6). The pre-processing module 22 then superimposes the second eye orbit image to the third eye orbit image to generate a superimposed orbit image (as shown in FIG. 3-7). On the other hand, the eye orbit images are normalized photographs, and the first eye orbit image may also be used as the training input data for the deep learning models of MRD1 and MRD2, and the superimposed eye orbit images (as shown in FIG. 3-7) may also be used as the training input for the deep learning model of LF.

In addition, the pupil coordinate regression model is a model trained by constructing a MobilenetV2 model, which enables the pupil coordinate regression model to learn from the first eye image, the second eye image and the third eye image to find the position of corneal light reflex. In one embodiment, the eye measure prediction module 23 in the cloud processing device 20 includes a prediction model, and the prediction model is a deep learning model, for example, a convolutional neural network (CNN) model, wherein the eye measure prediction module 23 receives the first eye orbit image and the superimposed eye orbit image, and uses the prediction model to calculate the user's MRD1 and MRD2 based on the first eye orbit image, and LF based on the superimposed eye orbit image.

In one embodiment, the establishment of the prediction model of the eye measure prediction module 23 is described as follows.

1. Establishment of photographs and gold standard measure (actual measure): a scale of 20×20 millimeters (mm) is placed on the dorsum of the subject's nose as a reference. To be clear, this scale is only necessary for gold standard measure while not for deep learning model training or for determining the model's accuracy. Next, a photographing device (such as a smartphone or a camera, etc.) takes the eye orbit photographs of the subjects' bilateral eyes (the subject is standing or sitting; a total of 6 photos, including viewing forward, upward gaze and downward gaze), and the shooting position is about 20-30 centimeters (cm) away from the subject's eyes. The shooting device and the subject's eyes are at the same level, thereby simulating the distance between the patient and the doctor when MRD1, MRD2, LF are manually measured by the doctors with a hand-held ruler in the clinic.

Furthermore, the photographs of the eye orbits taken are enlarged on the computer to obtain the measures of MRD1, MRD2 and LF manually by a plurality of doctors. The measures obtained by the plurality of doctors are averaged to respectively obtain the gold-standard measurement values (actual values) of MRD1, MRD2 and LF, followed by generating a plurality of labels according to the gold-standard measurement values (actual values) of MRD1, MRD2 and LF to serve as input data for deep learning model training. In addition, all MRD1 measures in upper eyelid ptosis without conical light reflex (i.e. pupillary light reflex) are set to 0. 2. Establishment of the prediction model: the prediction model of the eye measure prediction module 23 includes SENet (Squeeze-and-Excitation Networks, compression and excitation network) and Convolutional Neural Network (CNN) models such as EfficientNet. In detail, FIG. 4 is a flowchart illustrating the training process of the prediction model of the eye measure prediction module 23. As shown in FIG. 4, the training of the prediction model of the eye measure prediction module 23 includes the following steps:

(1) Input whole face images of the plurality of subjects and the plurality of labels to the eye measure prediction module 23, wherein the whole face images of the plurality of subjects include primary-gaze (eyes viewing forward) images and images of the eyes gazing up and down. In one embodiment, the eye measure prediction module 23 carries out a model training with minibatches mode, such as: input thirty two training images at a time, wherein the minibatch is selected upon the maximum number that can be processed according to the performance of the memory consumption and Graphics Processing Unit (GPU).

(2) The eye measure prediction module 23 confirms the whole face images of the plurality of subjects (the file format can be .PNG) and the plurality of labels (the file format can be .CSV), and the plurality of labels are mapping to the whole face images of the plurality of subjects.

(3) The eye measure prediction module 23 performs data pre-processing upon the whole face images of the plurality of subjects to crop the whole face images of the plurality of subjects into the first eye orbit images, the second eye orbit images and the third eye orbit images of the plurality of subjects, respectively, and then the second eye orbit images and the third eye orbit images of the plurality of subjects are superimposed to generate the superimposed eye orbit images of the plurality of subjects, so that the first orbit images and the superimposed eye orbit images of the plurality of subjects are used as a plurality of training images, wherein the first eye orbit images are used as the training input data for the prediction model to predict MRD1 and MRD, and the superimposed eye orbit images are used as the training input data for the prediction model to predict the LF, which further includes the following steps:

(3-1) The eye measure prediction module 23 uses the bilinear interpolation method to adjust the plurality of training images to 256×256 pixels.

(3-2) The eye measure prediction module 23 performs horizontal flip on the plurality of training images and imposes the separation method to rotate them randomly to increase the amount of data during training.

(3-3) The five-fold cross-validation of the eye measure prediction module 23 is used to estimate the performance of the model, and to divide the plurality of training images into five equal parts, wherein four parts are used for training and one part is used for validation.

(4) The eye measure prediction module 23 utilizes the plurality of training images after pre-processing to train the prediction model, wherein the prediction model is established based on EfficientNet in combination with SENet. The architecture diagram illustrating the prediction model is shown in FIG. 4-1. Through assigning corresponding weights to each pixel of the plurality of training images based on the plurality of labels by EfficientNet and SENet, and through a large number of training images and the plurality of their corresponding labels, the rules could be captured by EfficientNet and SENet and thereby the prediction model be trained.

Furthermore, the eye measure prediction module 23 sets the dropout rate to 0.25 to 0.5 for regularization, and the learning rate is set based on cosine annealing and one-cycle policy strategy to adjust the step size of the prediction model training, and thereby to regulate the learning paces. Finally, the average output of the models is used to integrate EfficientNet and SENet to obtain more accurate results, and to minimize the deviation of the prediction errors to improve the prediction accuracy of the prediction model in the eye measure prediction module 23.

In one embodiment, the present disclosure uses a parameter optimized method to optimize the prediction model. For instance, the training process of the prediction model is optimized by AdamW of the Neural Weight Optimizer using weight decay and L2 regularization, wherein L2 regularization will add a penalty term composed of the square of all weights in the prediction model to the Loss Function, and combine with specific hyperparameters to control the penalty. Furthermore, the Loss Function used in the present disclosure is MSE (mean square error) Loss, so that the prediction model described herein can be judged through the Loss Function.

Further, the prediction model of the eye measure prediction module 23 is established by SENet in combination with EfficientNet, wherein EfficientNet is a fast and high-precision model, using a model constructed by depth, width and the resolution of the image. EfficientNet is adjusted via depth, width and resolution.

In one embodiment, by using the resolution of the plurality of training images, it is helpful to capture fine features. At the same time, the accuracy of the model can be improved. Furthermore, by increasing the depth of EfficientNet (that is, increasing the number of convolutional layers), various complex features in the inputted plurality of training images can be found, and complex problems can be dealt. Ability of generalization is also better. However, the deeper the depth of EfficientNet, the more difficult it is to train the model, and its effect on model accuracy decreases as the depth increases. On the other hand, fine-grained features can be found by adjusting the width, which also makes the model easier to train. However, a model that is too wide will increase the difficulty of extracting higher level features, and the increase in model accuracy will soon saturate with width (that is, the accuracy will no longer improve).

In this regard, the eye measure prediction module 23 is based on EfficientNet in combination with SENet, to significantly establish the interdependence between the features and channels via SENet, and to adopt a whole new “feature reweight” strategy. Specifically, the feature re-calibration strategy is for SENet to automatically obtain the importance of each feature and channel via deep learning, so as to improve the useful features according to the importance of each feature and channel, and suppress the current task with a feature that is of little use. Therefore, the core module of SENet learns the feature weight via the network and the loss, so that the feature weight of effective features is enlarged, and the feature weight of invalid or insignificant features is reduced. In this way, the core model of SENet is trained. In order to better distinguish the importance of each feature and channel, the feature weights adjusted by SENet are used to improve the accuracy of EfficientNet.

(5) The eye measure prediction module 23 completes the training of the prediction model.

Therefore, after the above-mentioned training, the prediction model of the eye measure prediction module 23 can accurately calculate MRD1 and MRD2 according to the first eye orbit image of the subject, as well as the superimposed eye orbit images of the subject to calculate LF.

In one embodiment, the data transmission module 21 sends MRD1, MRD2 and LF of the user back to the measurement application 10a of the client device 10 for use in an analysis report interface (such as FIG. 2-30) to display the predicted measure of MRD1, MRD2 and LF, and MRD1, MRD2 and LF are provided to the clinicians as the basis for subsequent medical diagnosis.

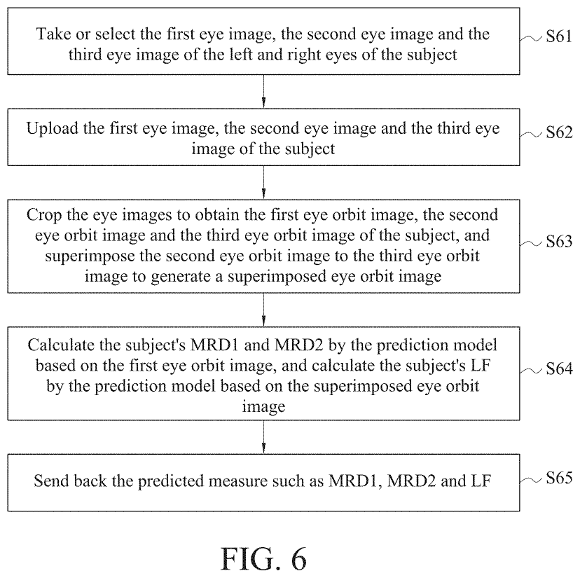

FIG. 6 is a flowchart illustrating the eye measurement method of the present disclosure, and is described with reference to FIG. 1 and FIG. 2-13 to FIG. 2-28, wherein the method includes the following steps S61 to S65:

In step S61, a doctor or patient uses the client device 10 with the measurement application 10a to capture the first eye image of the patient's left and right eyes, the second eye image (the image of the left and right eyes gazing up), and the third eye image (the image of the left and right eyes gazing down), or selects the stored images of the first eye image, the second eye image and the third eye image of the patient from the client device 10.

In step S62, the measurement application 10a uploads the first eye image, the second eye image and the third eye image of the patient to a cloud processing device 20.

In step S63, the cloud processing device 20 receives the first eye image, the second eye image and the third eye image of the patient. The first eye image, the second eye image, and the third eye image are pre-processed via cropping into the rectangular first eye orbit image, the second eye orbit image and the third eye orbit image, and the second eye orbit image is superimposed to the third eye orbit image to produce a superimposed eye orbit image.

In step S64, the cloud processing device 20 uses a prediction model to calculate MRD1 and MRD2 of the patient according to the first eye orbital image, and uses the prediction model to calculate LF according to the superimposed eye orbit image.

In step S65, the cloud processing device 20 sends the predicted measures such as MRD1, MRD2 and LF to the measurement application 10a of the client device 10, so as to present the predicted measures to the physician, enabling the physician to diagnose the patient based on the predicted measures.

In addition, the present disclosure also discloses a computer-readable medium, which is applied to a computing device or computer having a processor (e.g., CPU, GPU, etc.) and/or memory, and stores instructions, and utilizes the computing device or computer to execute the computer-readable medium via a processor and/or memory so as to execute the above-mentioned methods and steps when executing the computer-readable medium.

The following is an embodiment of the present disclosure, and is described with reference to FIG. 1 to FIG. 6. In addition, the embodiment is the same as the above-mentioned embodiment and will not be repeated.

In an embodiment, during clinical practice, the physician uses a smartphone (i.e., the client device 10) with the measurement application 10a to take a first eye image of the left and right eyes of a patient (as shown in the FIG. 3-1, the image of the left and right eyes viewing forward), a second eye image (as shown in FIG. 3-3, the image of the left and right eyes gazing up) and a third eye image (as shown in FIG. 3-5, the images of the left and right eyes gazing down). The physician uses the measurement application 10a in the smartphone to upload the patient's first eye image, second eye image and the third eye image to a cloud processing device 20. Alternatively, the patient can also use the smartphone to capture the first eye image, the second eye image and the third eye image of his/her left and right eyes, and the patient can also use the smartphone with the measurement application 10a to upload the first eye image, the second eye image and the third eye image to the cloud processing device 20.

After the cloud processing device 20 receives the first eye image, the second eye image and the third eye image of the patient, the pre-processing module 22 in the cloud processing device 20 pre-processes the first eye image, the second eye image and the third eye image by cropping the first eye image, the second eye image and the third eye image into the first eye orbit image (as shown in FIG. 3-2), the second eye orbit image (as shown in FIG. 3-4) and the third eye orbit image (as shown in FIG. 3-6), respectively. The pre-processing module 22 then superimposes the second eye orbit image to the third eye orbit image to generate a superimposed eye orbit image (as shown in FIG. 3-7).

Afterwards, the eye measure prediction module 23 in the cloud processing device 20 uses its prediction model to calculate MRD1 and MRD2 according to the patient's first eye orbital image, and calculates LF according to the patient's superimposed eye orbit image. The data transmission module 21 in the cloud processing device 20 sends the patient's MRD1, MRD2 and LF back to the measurement application 10a of the smartphone, so that the measurement application 10a displays the predicted measures of MRD1, MRD2 and LF via the analysis report interface (as shown in FIG. 2-27), and the predicted measures of MRD1, MRD2 and LF are provided to the physician as a basis for subsequent medical diagnosis.

To sum up, the eye measurement system, the method and the computer-readable medium of the present disclosure receive, via the cloud processing device, the subject's first eye image (left eye and/or right eye viewing forward), second eye image (left eye and/or right eye gazing upward), and third eye image (left eye and/or the right eye gazing down) uploaded by a clinician or a subject himself/herself using a measurement application in the client device. After pre-processing by the cloud processing device, the first eye orbit image and the superimposed eye orbit image of the patient are obtained, so that the prediction model can obtain MRD1, MRD2 and LF according to the first eye orbit image and the superimposed eye orbit image, and provide MRD1, MRD2 and LF to the clinicians as a basis for diagnosis. Therefore, as compared with the prior art, the present disclosure does not confine to the places where the eye images are taken, and the prediction model can accurately obtain the subject's eye measures, thereby providing doctors with a clear diagnosis basis.

In addition, the eye measurement system, the method and the computer-readable medium of the present disclosure have the following advantages or technical effects.

1. The present disclosure utilizes the deep learning prediction model to measure MRD1, MRD2 and LF on close-up eye images (such as eye orbit photos). In addition to record the subjects' eye state in detail, through such prediction model, the accuracy of measurement can be improved, and the detection time is reduced, which improves the efficiency of clinics.

2. Through the measurement application designed in the present disclosure, a device such as a smartphone can be used to measure the predicted value of the patient's eye in any occasion, and the measurement application can use the smartphone to measure the predicted measures of the eye. The patient's eye photos taken by the smartphone are transmitted to the cloud processing device, and the artificial intelligence deep learning module can be used to automatically analyze the eye images, accurately analyze the patient's eye state, and the obtained predicted measures (MRD1, MRD2 and LF value) are then sent back to the client device to provide the clinicians as a basis for diagnosis.

3. The measurement application designed by the present disclosure can help to record the patient's eye images, and receive reminders and health-related information provided by the healthcare providers, thereby improving the quality and efficiency of patient care.

4. The present disclosure provides users (such as patients, their family members or healthcare providers, etc.) account management functions (such as login, password change, etc.) and patient information management functions via the user interface presented by the measurement application (e.g., patient information, eye monitoring, eye operation information, etc.) and notification management functions (e.g., reminder notification, health-related information, etc.), giving the users a good user experience of the application, and providing a complete application function.

5. The present disclosure utilizes the pupil coordinate regression model to confirm the corneal light reflex position in the eye image, thereby automatically cropping the first eye image, the second eye image and the third eye image based on the corneal light reflex position to obtain the first eye orbit image, the second eye orbit image and the third eye orbit image, which can be used as the source of measurement, or as the input materials for the training of the prediction model.

6. The prediction model of the present disclosure is based on EfficientNet and is established in combination with SENet. Through SENet, the interdependence between features and channels is significantly established to improve the feature weight of effective features, and reduce the feature weights of invalid or insignificant features, thereby providing adjusted feature weights to EfficientNet. Therefore, EfficientNet can significantly improve its prediction accuracy by adjusting the feature weights.

The above-mentioned embodiments are illustrations of the principles and effects of the present disclosure, but are not intended to limit the present disclosure. Any person skilled in the art can modify and change the above-mentioned embodiments without departing from the spirit and scope of the present disclosure. Therefore, the protection scope of the present disclosure should be listed in the claims of the present disclosure.

What is claimed is:

1. An eye measurement system, comprising:
a client device with a measurement application, configured for capturing a first eye image, a second eye image and a third eye image of a subject, or for selecting the first eye image, the second eye image and the third eye image of the subject from the client device; and
a cloud processing device, communicatively connected to the client device, configured for receiving the first eye image, the second eye image and the third eye image of the subject from the measurement application, comprising:
a pre-processing module to crop the first eye image, the second eye image and the third eye image of the subject into a first eye orbit image, a second eye orbit image and a third eye orbit image, respectively, and superimposing the second eye orbit image to the third eye orbit image to generate a superimposed eye orbit image; and
an eye measure prediction module with a prediction model to calculate a predicted eye measure based on the first eye orbit image and the superimposed eye orbit image of the subject, wherein the cloud processing device sends the predicted eye measure of the subject back to the measurement application for supplying the predicted eye measure.

2. The eye measurement system according to claim 1, wherein the first eye image is a photograph of the subject's left and right eyes viewing forward, the second eye image is a photograph of the subject's left and right eyes gazing up, and the third eye image is a photograph of the subject's left and right eyes gazing down.

3. The eye measurement system according to claim 1, wherein the predicted eye measure includes an MRD1, an MRD2 and an LF, wherein the MRD1 and the MRD2 are calculated from the first eye orbit image and the LF is calculated from the superimposed eye orbit image by the prediction model of the eye measure prediction module.

4. The eye measurement system according to claim 1, wherein the pre-processing module uses a coordinate regression model to determine a position of a conical light reflex of the first eye image, the second eye image and the third eye image of the subject, and crops the first eye image, the second eye image and the third eye image into the first eye orbit image, the second eye orbit image and the third eye orbit image based on the position of the corneal light reflex.

5. The eye measurement system according to claim 1, wherein the measurement application includes a data management module for viewing the subject's data, information on eye operation, or monitoring the subject's eye condition.

6. The eye measurement system according to claim 1, wherein the measurement application includes a notification management module for receiving health-related information and notifications, managing the subject's groups, or displaying a message sending history.

7. The eye measurement system according to claim 1, wherein the prediction model is established based on an EfficientNet in combination with an SENet, the eye measure prediction module inputs a plurality of training images into the prediction model to perform a deep learning, and the prediction model calculates the predicted eye measure of the subject based on the first eye orbit image and the superimposed eye orbit image after finishing the deep learning, wherein the SENet increases feature weights of significant features and reduces feature weights of invalid or insignificant features in the plurality of training images, such that the EfficientNet performs the deep learning based on adjusted feature weights and the plurality of training images.

8. An eye measurement method, comprising:
capturing a first eye image, a second eye image and a third eye image of a subject by a client device with a measurement application, or selecting the first eye image, the second eye image, and the third eye image of the subject from the client device by the measurement application;
receiving the first eye image, the second eye image and the third eye image of the subject from the measurement application by a cloud processing device;
cropping the first eye image, the second eye image and the third eye image of the subject into a first eye orbit image, a second eye orbit image and a third eye orbit image by the cloud processing device, respectively, wherein the second eye orbit image is superimposed to the third eye orbit image to generate a superimposed eye orbit image;
using a prediction model by the cloud processing device to calculate a predicted eye measure of the subject based on the first eye orbit image and the superimposed eye orbit image; and
sending the predicted eye measure of the subject back to the measurement application by the cloud processing device for supplying the predicted eye measure.

9. The eye measurement method according to claim 8, wherein the first eye image is a photograph of the subject's left and right eyes viewing forward, the second eye image is a photograph of the subject's left and right eyes gazing up, and the third eye image is a photograph of the subject's left and right eyes gazing down.

10. The eye measurement method according to claim 8, wherein the predicted eye measure includes an MRD1, an MRD2 and an LF, wherein the MRD1 and the MRD2 are calculated from the first eye orbit image and the LF is calculated from the superimposed eye orbit image by the prediction model of the eye measure prediction module.

11. The eye measurement method according to claim 8, further comprising using a coordinate regression model by the cloud processing device to determine a position of a corneal light reflex of the first eye image, the second eye image and the third eye image of the subject, and cropping the first eye image, the second eye image and the third eye image into the first eye orbit image, the second eye orbit image and the third eye orbit image based on the position of the corneal light reflex.

12. The eye measurement method according to claim 8, wherein the measurement application includes a data management module for viewing the subject's data, information on eye operation, or monitoring the subject's eye condition.

13. The eye measurement method according to claim 8, wherein the measurement application includes a notification management module for receiving health-related information and notifications, managing the subject's groups, or displaying a message sending history.

14. The eye measurement method according to claim 8, wherein the prediction model is established based on an EfficientNet in combination with an SENet, the eye measure prediction module inputs a plurality of training images into the prediction model to perform a deep learning, and the prediction model calculates the predicted eye measure of the subject based on the first eye orbit image and the superimposed eye orbit image after finishing the deep learning, wherein the SENet increases feature weights of significant features and reduces feature weights of invalid or insignificant features in the plurality of training images, such that the EfficientNet performs the deep learning based on adjusted feature weights and the plurality of training images.

15. A computer-readable medium, applied on a computing device or a computer, stores instructions for executing the eye measurement method according to claim 8.
